(19) United States
(12) Patent Application Publication
(10) Pub. No.: US 2024/0316555 A1
(43) Pub. Date: Sep. 26, 2024

(54) DEVICE FOR FILTERING SPECIFIC CELLS

(71) Applicant: GENERAL BIOLOGICALS CORPORATION, Hsinchu County (TW)

(72) Inventors: Pankaj Kumar Joshi, Hsinchu County (TW); Leo Liu, Hsinchu County (TW); Frank Lin, Hsinchu County (TW); Tsong Chin Lin, Hsinchu County (TW)

(21) Appl. No.: 18/736,564

(22) Filed: Jun. 7, 2024

Related U.S. Application Data
(63) Continuation-in-part of application No. 17/037,731, filed on Sep. 30, 2020, now abandoned.

(51) Int. Cl. B01L 3/00

(52) U.S. Cl. CPC B01L 3/502761; B01L 2200/027; B01L 2200/0652; B01L 2300/0681; B01L 2300/0832

(57) ABSTRACT

A device for filtering specific cells includes an upper frame, an upper frame, and a flexible transparent filtration membrane. The upper frame has a first outer peripheral portion and a first inner peripheral portion. The first inner peripheral portion forms an inlet and a first cavity communicating with the inlet. The lower frame has a second outer peripheral portion and a second inner peripheral portion. The second inner peripheral portion forms an outlet and a second cavity communicating with the outlet. The flexible transparent filtration membrane is detachably connected to the first cavity and the second cavity.

### CROSS REFERENCE TO RELATED APPLICATIONS

This is a continuation-in-part application of co-pending U.S. patent application Ser. No. 17/037,731, “MICROFILTER, MANUFACTURING METHOD AND MICROFILTRATION UNIT”, filed on Sep. 30, 2020.

### BACKGROUND OF THE INVENTION

### 1. Field of the Invention

The present invention relates to a filtration device, and more particularly to a device for filtering specific cells.

### 2. Description of the Prior Art

The microfilter structure may be a solid membrane structured with microholes to make it a semi-permeable medium. The microfilter structure has been commonly applied to separate micro objects from complex samples.

A large variety of materials have been used to manufacture microfilters such as polymers, metals or composites depending on the field of application. For example, the epoxy based microfilms have been commonly used in biomolecules isolation. Steel based microfilters are common in inkjet nozzles.

The microfilter structure has been employed in various fields of applications such as in electronic, chemical, food and biotechnology industries. Examples of the microfilter application include size classification of solid objects between micro and macromolecules using microsieves, micro particles isolation from liquids in biomedical filtration or refining processes, uniform distribution of liquid or gas inlet for micro spray generation in printing or drug delivery and an MEMS structure fabrication making use of a deposition mask.

The biomolecules such as bacteria, viruses, cells, vesicles, etc. possess structural flexibility. For a particular target, a precision microfilter is required to optimize the membrane pressure. A uniform array of cylindrical microholes may give the efficient filtration yield and smooth biomolecules isolation from various body fluids such as saliva, blood, urine, dialysis fluid etc.

A conventional microfilter structure includes a frame and a filtration membrane fixed in the frame. The filtration membrane is made of a brittle material, which is easy to be ruptured by negative pressure during filtration. Besides, the filtration membrane cannot be taken out individually. Since the microfilter is not flat, it is required to use an instrument with a specific structure, such as a specific microscope with a holder on the carrier for observing and studying the biomolecules on the filtration membrane. It is inconvenient to use the microfilter. Therefore, it is necessary to improve the conventional microfilter structure.

### SUMMARY OF THE INVENTION

In view of the deficiencies of the prior art, the primary object of the present invention is to provide a device for filtering specific cells, which can effectively solve the problem that the filtration membrane of the conventional microfilter structure is easy to rupture and inconvenient to use.

In order to achieve the foregoing object, the present invention adopts the following technical solutions:

A device for filtering specific cells comprises an upper frame, an upper frame, and a flexible transparent filtration membrane. The upper frame has a first outer peripheral portion and a first inner peripheral portion. The first inner peripheral portion forms an inlet and a first cavity communicating with the inlet. The lower frame has a second outer peripheral portion and a second inner peripheral portion. The second inner peripheral portion forms an outlet and a second cavity communicating with the outlet. The flexible transparent filtration membrane is disposed between the first cavity and the second cavity. The flexible transparent filtration membrane is detachably connected to the first cavity and the second cavity.

Preferably, a bottom end of the first inner peripheral portion has a first support surface, and a bottom of the second cavity has a second support surface. The flexible transparent filtration membrane includes an inner membrane portion and an outer membrane portion extending outwardly from an edge of the inner membrane portion. The inner membrane portion has filtration holes. The outer membrane portion is secured between the first support surface and the second support surface.

Preferably, a first elastic gasket is provided on the first support surface, and a second elastic gasket is provided on the second support surface. The outer membrane portion is tightly clamped between the first elastic gasket and the second elastic gasket.

Preferably, the first support surface, the second support surface, the first elastic gasket and the second elastic gasket are formed a seal structure.

Preferably, internal threads are formed on an inner wall of the first cavity, and external threads are formed on an outer wall of the second inner peripheral portion. The external threads cooperate with and are threadedly connected to the internal threads.

Preferably, a connecting strip has an end connected to the first outer peripheral portion and another end connected to a connector. The connector is detachably connected to the inlet. The connector has an access hole.

Preferably, the connecting strip is a flexible strip. The end of the connecting strip is integrally formed with the first outer peripheral portion, and another end of the connecting strip is integrally formed with the connector.

Preferably, a plurality of first anti-slip grooves are formed on an outer wall of the first outer peripheral portion.

Preferably, a plurality of second anti-slip grooves are formed on an outer wall of a lower end of the second outer peripheral portion.

Compared with the prior art, the present invention has obvious advantages and beneficial effects. Specifically, it can be known from the foregoing technical solutions:

The flexible transparent filtration membrane can be elastically deformed to effectively avoid rupture due to negative pressure and prolong the service life. Besides, the flexible transparent filtration membrane is detachably connected to the first cavity and the second cavity, so that the flexible transparent filtration membrane can be taken out individually for directly observing the targets on the flexible transparent filtration membrane. There is no need to use an instrument with a specific structure, facilitating ease of use.

Embodiments of the present invention will now be described, by way of example only, with reference to the accompanying drawings.

### BRIEF DESCRIPTION OF THE DRAWINGS

FIG. 1 is a perspective view according to a preferred embodiment of the present invention;

FIG. 2 is a cross-sectional view according to the preferred embodiment of the present invention;

FIG. 3 is a perspective view of the upper frame according to the preferred embodiment of the present invention;

FIG. 4 is a cross-sectional view of the upper frame according to the preferred embodiment of the present invention;

FIG. 5 is a perspective view of the lower frame according to the preferred embodiment of the present invention;

FIG. 6 is a cross-sectional view of the lower frame according to the preferred embodiment of the present invention;

FIG. 7 is a perspective view according to the preferred embodiment of the present invention when in use; and

FIG. 8 is a cross-sectional view according to the preferred embodiment of the present invention when in use.

### DETAILED DESCRIPTION OF THE PREFERRED EMBODIMENTS

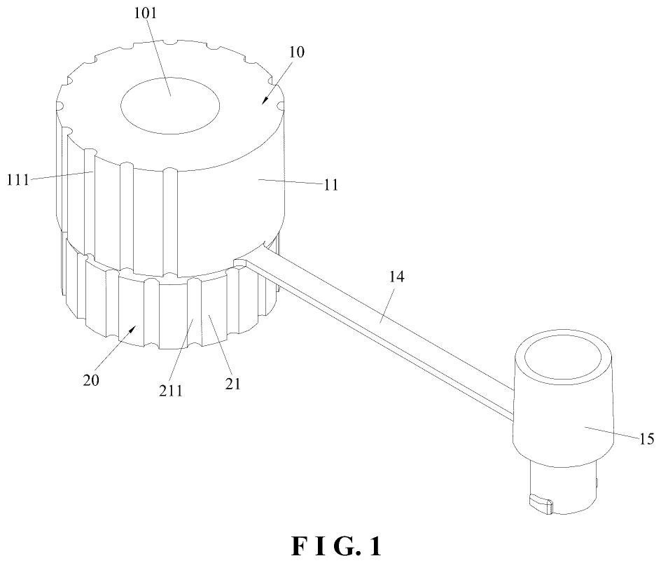

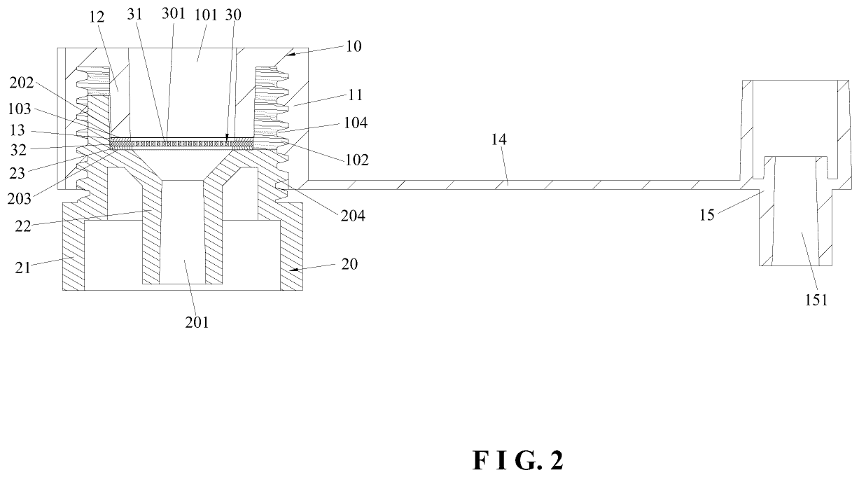

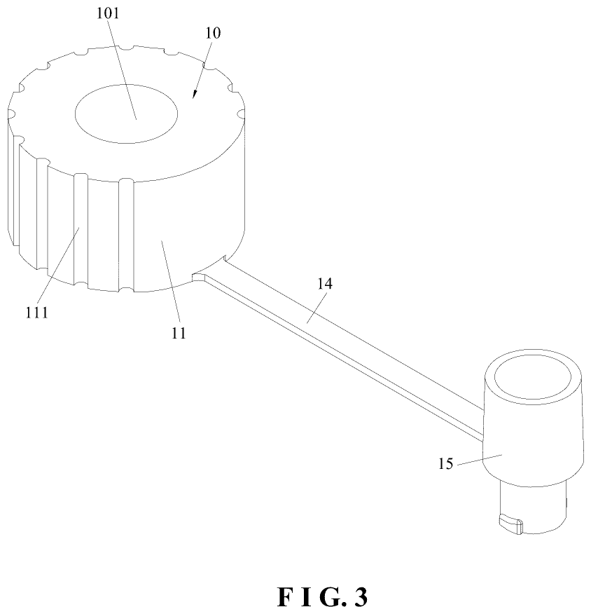

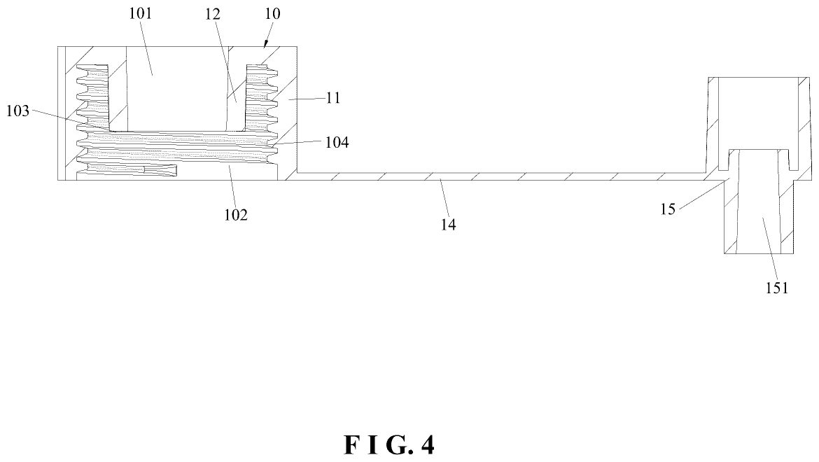

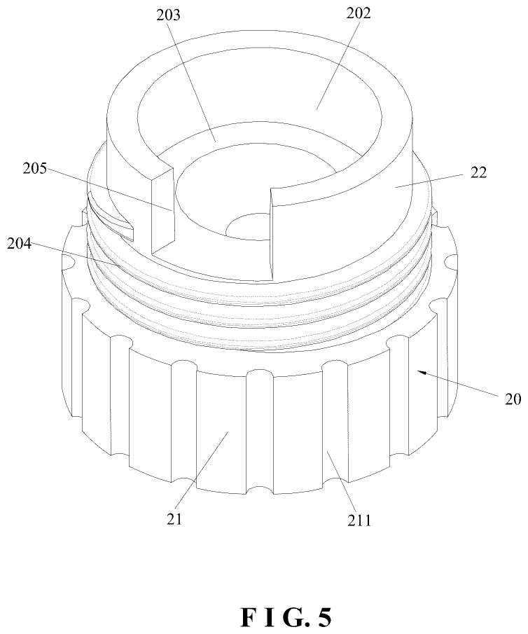

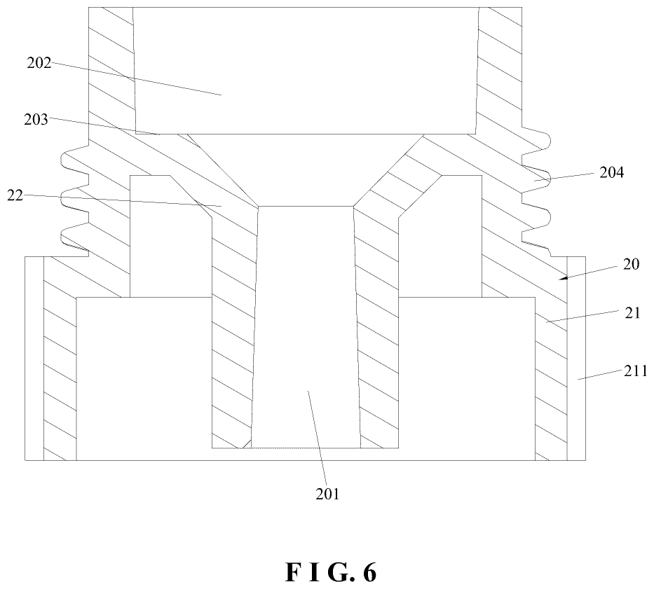

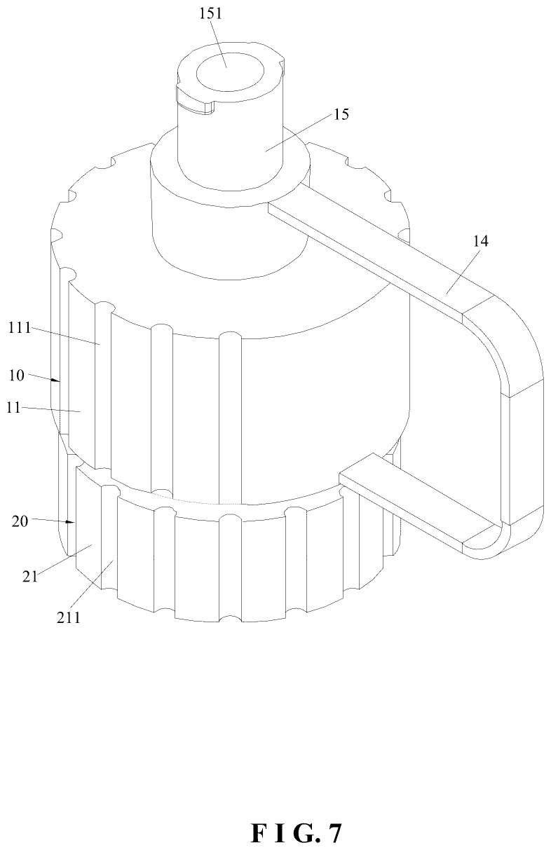

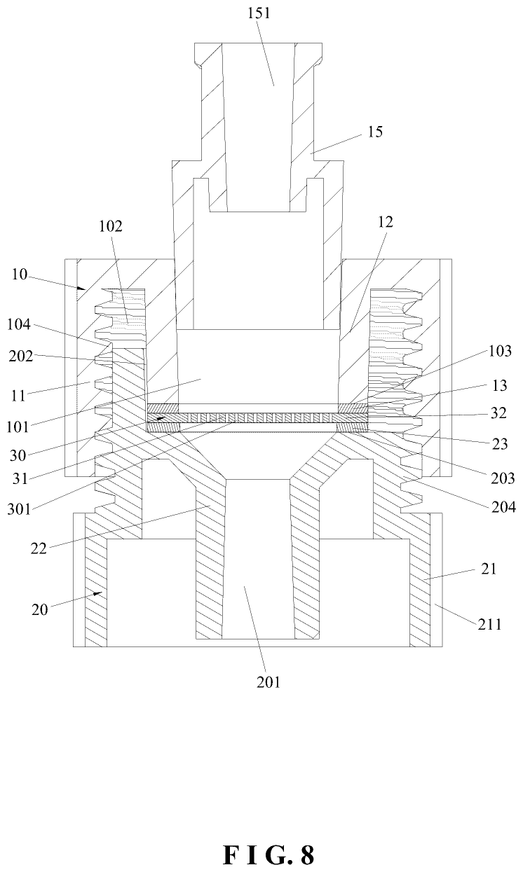

FIG. 1 to FIG. 8 illustrate the specific structure of a preferred embodiment of the present invention, comprising an upper frame 10, a lower frame 20, and a flexible transparent filtration membrane 30.

The upper frame 10 has a first outer peripheral portion 11 and a first inner peripheral portion 12. The first inner peripheral portion 12 forms an inlet 101 and a first cavity 102 communicating with the inlet 101. In this embodiment, a plurality of first anti-slip grooves 111 are formed on the outer wall of the first outer peripheral portion 11, which is beneficial for manual screwing. The bottom end of the first inner peripheral portion 12 has a first support surface 103. The first support surface 103 is annular. A first elastic gasket 13 is provided on the first support surface 103. The first elastic gasket 13 is annular and fit with the first support surface 103. The first elastic gasket 13 is made of a rubber material. Internal threads 104 are formed on the inner wall of the first cavity 102.

The lower frame 20 has a second outer peripheral portion 21 and a second inner peripheral portion 22. The second inner peripheral portion 22 forms an outlet 201 and a second cavity 202 communicating with the outlet 201. In this embodiment, a plurality of second anti-slip grooves 211 are formed on the outer wall of the lower end of the second outer peripheral portion 21, which is beneficial for manual screwing. The bottom of the second cavity 202 has a second support surface 203. The second support surface 203 is annular. A second elastic gasket 23 is provided on the second support surface 203. The second elastic gasket 23 is annular and fit with the second support surface 203. The second elastic gasket 23 is made of a rubber material. External threads 204 are formed on the outer wall of the second inner peripheral portion 22. The external threads 204 cooperate with and are threadedly connected to the internal threads 104. The assembly and disassembly of the device are fast and easy. The upper end of the second inner peripheral portion 22 has a notch 205. The notch 205 communicates with the second cavity 202, which facilitates removal of the flexible transparent filtration membrane 30 from the second cavity 202.

The flexible transparent filtration membrane 30 is disposed between the first cavity 102 and the second cavity 202. The flexible transparent filtration membrane 30 is detachably connected to the first cavity 102 and the second cavity 202. The flexible transparent filtration membrane 30 is used for filtering or isolating targets. In this embodiment, the flexible transparent filtration membrane 30 includes an inner membrane portion 31 and an outer membrane portion 32 extending outwardly from the edge of the inner membrane portion 31. The inner membrane portion 31 has filtration holes 301. The diameter of the filtration holes 301 depends on the size of the screening targets. The shape of the filtration holes 301 may be circular, rectangular or hexagonal, depending on the screening targets. The filtration holes 301 are patterned in a uniform array in a straight, staggered or hexagonal geometry in the inner membrane portion 31. The filtration holes 301 in the inner membrane portion 31 have cylindrical walls or tapered walls. The number of the filtration holes 301 is not less than 50,000, and the gap between two adjacent filtration holes 301 is optimized at about 20 μm. The filtration holes 301 are distributed in different sections. The outer membrane portion 32 is secured between the first support surface 103 and the second support surface 203. The outer membrane portion 32 is tightly clamped between the first elastic gasket 13 and the second elastic gasket 23 to avoid sample leakage and to secure the flexible transparent filtration membrane 30 in place. The thickness of the flexible transparent filtration membrane 30 is less than or equal to 25 μm. The diameter of the flexible transparent filtration membrane 30 is 13 mm. The diameter of the inner membrane portion 31 is 9 mm. The flexible transparent filtration membrane 30 is fabricated by perforating a non-epoxy-based microfilm using different lasers such as UV, visible and Infrared lasers. The micro filtration holes 301 are formed by using the above lasers, the shorter wavelength provides the filtration holes 301 with good quality and a submicron diameter. The excimer lasers, such as Krf & XeBr, etc., provide a fine cylindrical filtration hole structure. The flexible transparent filtration membrane 30 is made of polyimide, polycarbonate, polyethylene, polyethylene terephthalate, polyethylene naphthalate, polymethyl methacrylate and polystyrene.

Furthermore, a connecting strip 14 has an end connected to the first outer peripheral portion 11 and another end connected to a connector 15. The connector 15 is detachably connected to the inlet 101. The connector 15 has an access hole 151. The connector 15 may be replaced with a different one, so as to correspondingly mate with an external sample inlet syringe with a different sample inlet orifice for the sample to be injected from that external sample inlet syringe. In this embodiment, the connecting strip 14 is a flexible strip. The end of the connecting strip 14 is integrally formed with the first outer peripheral portion 11, and another end of the connecting strip 14 is integrally formed with the connector 15, which facilitates use and avoids loss of the connector 15. The connecting strip 14 may be detachably connected to the lower end of the first peripheral portion 11 or the connector 15 through a male/female coupler (not shown), such as Velcro or other connecting unit.

The use of this embodiment is described in detail as follows:

When in use, the flexible transparent filtration membrane 30 is placed between the upper frame 10 and the lower frame 20, and the upper frame 10 and the lower frame 20 are inserted and connected to each other, so that the flexible transparent filtration membrane 30 is secured between the first cavity 102 and the second cavity 202. Then, the outlet 201 is connected to a controlled suction pump. Negative pressure is applied to the second cavity 202 using the suction pump. An external sample inlet syringe is inserted in the inlet 101 for injecting the sample into the first cavity 102 and onto the flexible transparent filtration membrane 30. Under the action of negative pressure, the non-targets pass through the filtration holes 301 and are output from the outlet 201, and the targets are isolated on the flexible transparent filtration membrane 30. Next, the upper frame 10 and the lower frame 20 are separated from each other, and the flexible transparent filtration membrane 30 is taken out. Finally, the flexible transparent filtration membrane 30 is moved to other instruments for analyzing and studying the targets on the flexible transparent filtration membrane 30.

The device provided by the present invention may be used in medical diagnosis and prognosis. The flexible transparent filtration membrane 30 can isolate larger nucleated cells from the blood. The enumeration and collection of nucleated cell indicates the disease state. The isolated cells are further utilized for downstream analysis such as culture, immunostaining, PCR (Polymer chain reaction), NGS (Next-Generation sequencing), etc. The device provided by the present invention may be used for non-invasive tumor metastasis diagnosis by identifying the circulating tumor cells (CTCs). The tumor cells are larger in size than normal cells. The tumor cells are isolated by passing the blood through the flexible transparent filtration membrane 30. The isolated CTCs on the flexible transparent filtration membrane 30 are identified and enumerated by immunofluorescent staining and observed under fluorescence microscope.

The flexible transparent filtration membrane 30 of the device may be used for non-invasive prenatal diagnosis by collecting colony-forming cells (CFCs) from the maternal blood. The CFC cells are bigger than normal blood components. The isolated CFCs are analyzed for genetic analysis by using FISH, NGS, etc.

The flexible transparent filtration membrane 30 may be used for separating micro aggregates from the blood. In particular, preserved blood may have micro aggregates during storage. The device provided by the present invention is used to clear the micro aggregates before blood transfusion to a patient.

The feature of the present invention is that the flexible transparent filtration membrane can be elastically deformed to effectively avoid rupture due to negative pressure and prolong the service life. Besides, the flexible transparent filtration membrane is detachably connected to the first cavity and the second cavity, so that the flexible transparent filtration membrane can be taken out individually for directly observing the targets on the flexible transparent filtration membrane. The flatness is guaranteed. There is no need to use an instrument with a specific structure, facilitating ease of use.

Although particular embodiments of the present invention have been described in detail for purposes of illustration, various modifications and enhancements may be made without departing from the spirit and scope of the present invention. Accordingly, the present invention is not to be limited except as by the appended claims.

What is claimed is:

1. A device for filtering specific cells, comprising:
an upper frame, having a first outer peripheral portion and a first inner peripheral portion, the first inner peripheral portion forming an inlet and a first cavity communicating with the inlet;
a lower frame, having a second outer peripheral portion and a second inner peripheral portion, the second inner peripheral portion forming an outlet and a second cavity communicating with the outlet;
a flexible transparent filtration membrane, disposed between the first cavity and the second cavity;
wherein the flexible transparent filtration membrane is detachably connected to the first cavity and the second cavity.

2. The device as claimed in claim 1, wherein a bottom end of the first inner peripheral portion has a first support surface, a bottom of the second cavity has a second support surface, the flexible transparent filtration membrane includes an inner membrane portion and an outer membrane portion extending outwardly from an edge of the inner membrane portion, the inner membrane portion has filtration holes, and the outer membrane portion is secured between the first support surface and the second support surface.

3. The device as claimed in claim 2, wherein a first elastic gasket is provided on the first support surface, a second elastic gasket is provided on the second support surface, and the outer membrane portion is tightly clamped between the first elastic gasket and the second elastic gasket.

4. The device as claimed in claim 3, wherein the first support surface, the second support surface, the first elastic gasket and the second elastic gasket are formed a seal structure.

5. The device as claimed in claim 1, wherein internal threads are formed on an inner wall of the first cavity, external threads are formed on an outer wall of the second inner peripheral portion, and the external threads cooperate with and are threadedly connected to the internal threads.

6. The device as claimed in claim 1, wherein a connecting strip has an end connected to the first outer peripheral portion and another end connected to a connector, the connector is detachably connected to the inlet, and the connector has an access hole.

7. The device as claimed in claim 6, wherein the connecting strip is a flexible strip, the end of the connecting strip is integrally formed with the first outer peripheral portion, and another end of the connecting strip is integrally formed with the connector.

8. The device as claimed in claim 1, wherein a plurality of first anti-slip grooves are formed on an outer wall of the first outer peripheral portion.

9. The device as claimed in claim 1, wherein a plurality of second anti-slip grooves are formed on an outer wall of a lower end of the second outer peripheral portion.
